(19) United States
(12) Patent Application Publication
(10) Pub. No.: US 2002/0195110 A1
(43) Pub. Date: Dec. 26, 2002

(54) Medico-surgical tubes

(76) Inventor: Joan Elizabeth Watton, Kent (GB)

Correspondence Address: LOUIS WOO, LAW OFFICE OF LOUIS WOO, 1901 NORTH FORT MYER DRIVE, SUITE 501, ARLINGTON, VA, 22209, US

(21) Appl. No.: 10/142,979

(22) Filed: May 13, 2002

(30) Foreign Application Priority Data
Jun. 22, 2001 (GB) 0115327.9
Aug. 17, 2001 (GB) 0120102.9

(51) Int. Cl. A62B 7/00; A61M 16/00

(52) U.S. Cl. 128/207.15; 128/204.18

(57) ABSTRACT

An endotracheal tube has a shaft extruded of a plastics material with a lumen within its wall. A rod of a malleable metal extends along the lumen and is spaced from opposite ends of the tube. The tube can be bent to retain a desired bend where it emerges from the patient's mouth.

### BACKGROUND OF THE INVENTION

This invention relates to medico-surgical tubes.

Endotracheal tubes have a patient end located, in use, within the trachea and a machine end that emerges from the mouth or nose of the patient. Ventilation tubing is connected to the machine end of the tube, which may be cut so that it does not project very far beyond the mouth or nose. Although the ventilation tubing is flexible, it still projects some distance from the patient, which can clutter the facial region and apply leverage to the tracheal tube. It has been proposed that tracheal tubes be bent sharply where they emerge from the nose or mouth so that the machine end of the tube lies along the surface of the face. The machine end of the tube may be bent up, cephaladly, or down, caudadly. Such tubes are described, for example, in U.S. Pat. No. 3,964,488. Tubes of this kind are sold by Mallinckrodt Inc under the name “RAE” and by Portex Limited under the name “Polar”. Such tube have an advantage in that the relatively bulky ventilation tubing can be connected at some distance from the mouth and it lies more closely against the patient. One problem with such tubes, however, is that the bend in the tube may not be in the ideal position for all patients.

### BRIEF SUMMARY OF THE INVENTION

It is an object of the present invention to provide an alternative medico-surgical tube.

According to the present invention there is provided a medico-surgical tube of a first material, the tube including a separate member of a second, different material extending along a region of the length of the tube, and the second material being such that the tube can be bent by the user to a desired angle anywhere along the region with the tube retaining the angle of the bend.

The second material may be in the form of a rod of malleable metal extending longitudinally along the region. The second material may be a shape memory effect material. The region is preferably spaced from the patient end of the tube and may be spaced from the machine end of the tube. The first material is preferably a plastics. The tube may be an endotracheal tube and the region in use may extend partially within the body and partly outside the body, the tube being bent where it emerges from the body such that the machine end of the tube extends substantially parallel with the patient's anatomy. The tube may be extruded with a lumen within its wall, the second material extending along the lumen. The tube may be curved along its length, the second material extending along the inside of the curve of the tube. The tube may be reinforced with a helical reinforcement member.

An oral endotracheal tube according to the present invention will now be described, by way of example, with reference to the accompanying drawings.

### BRIEF DESCRIPTION OF THE DRAWINGS

FIG. 1 is a side elevation view of the tube;

FIG. 2 is an enlarged, transverse sectional view of the tube along the line II-II of FIG. 1; and

FIG. 3 shows the tube in use.

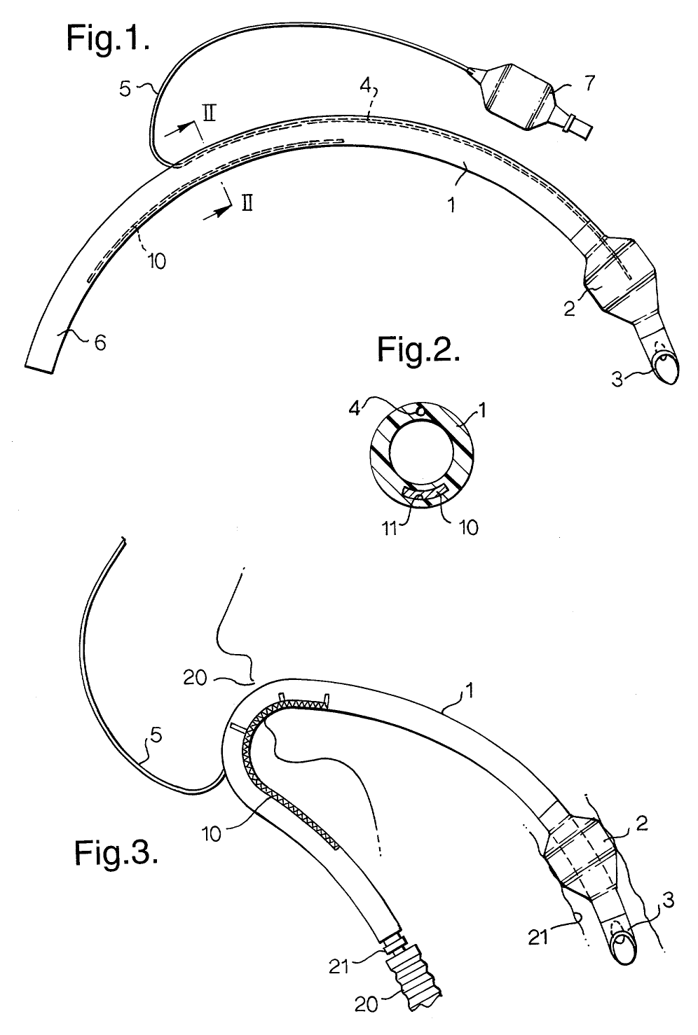

### DETAILED DESCRIPTION OF THE PREFERRED EMBODIMENT

The endotracheal tube has a shaft 1 of circular section and is curved along its length to the conventional shape of an endotracheal tube. The shaft 1 is extruded of PVC although it could be made of other plastics, such as polyurethane or silicone rubber, and it could be made in ways other than by extrusion. The tube has an inflatable cuff 2 embracing the shaft 1 close to its patient end 3. The interior of the cuff 2 communicates with a small-bore, minor lumen 4 extending along the tube, extruded within the wall of the shaft 1. The minor lumen 4 communicates with one end of an inflation line 5 towards the machine end 6 of the tube. The other end of the inflation line 5 is terminated with an inflation indicator and connector 7. As so far described, the tube is conventional.

The tube also includes an elongate member 10 of a malleable material, in the form of a metal rod, such as of iron or aluminium. The malleable rod 10 is embedded within the wall thickness of the shaft 1, such as by being located in a second, extruded minor lumen 11. On a typical adult tube, the rod extends from a point about midway along the tube towards the machine end 6 of the tube, terminating about 40 mm short of the machine end. The rod 10 is located on the inside of the preformed curve of the tube

The malleable rod 10 is inserted in the tube by pushing it into the lumen 11 after extrusion of the shaft 1, and retaining it in position such as by means of an adhesive applied to the outside of the rod. Alternatively, the lumen 11 could be blocked at opposite ends of the rod 10 by cement or the like. There are various other ways in which a malleable member 10 could be embedded in a tube. Short lengths of a malleable member could, for example be embedded during extrusion of the tube shaft 1 separated by spaces. A member in the form of a strip could be attached to an inner layer and subsequently covered by an outer layer.

In use, as shown in FIG. 3, the clinician inserts the patient end 3 of the tube through the mouth 20 to the correct position in the trachea 21, inflates the cuff 2 and then bends the tube either down (caudadly) or up (cephaladly), as desired, at some point along the length of the rod 10, usually where the tube emerges from the body, so that the machine end of the tube extends substantially parallel with the patient's anatomy. The length and position of the rod 10 is chosen so that the tube can be bent at the ideal location for all builds of patient with whom the tube of the particular size will be used. The rod 10 extends along a region that is partly within the body and partly outside the body. When bent in the region of the rod 10, the tube will retain the shape to which it is bent, until bent back. The machine end 6 of the tube can then be connected to ventilation tubing 20 using a conventional connector 21. Because most of the length of the tube within the patient is free of the malleable rod, it can maintain the thermo-softening properties and flexibility of a conventional plastics tube. The space between the machine end of the rod 10 and the machine end 6 of the tube enables the tube to be cut shorter, if desired. By ensuring that the machine end 6 is free of the malleable rod 10, a connector can be push fitted readily into the end of the tube. The tube could be reinforced along most of its length such as by a conventional helical reinforcing element. Such a reinforcing element is used in conventional tubes to give the tubes resistance to crushing and kinking without making the tube significantly less flexible. The malleable member could be a strip, rod or of other section and could be formed of various malleable materials. The invention is not confined to endotracheal tubes but could be used in other medico-surgical tubes.

The present invention enables a tube to be bent at the ideal location. With tracheal tubes, this can reduce the risk of over insertion of the tube, where an inexperienced user inserts the tube until the preformed bent is in the desired position without taking care to ensure that the patient end is correctly located. Because the tube can easily be bent back to its original shape, it is possible to straighten the machine end of the tube, while the patient end is located in the trachea, in order to insert an instrument or the like within the bore of the tube.

The elongate member could be of a shape memory effect metal, that is, an alloy that undergoes a reversible solid-state change over a narrow temperature range. In particular, the metal would be arranged to change shape, and thereby change the shape of the tube, such as, as a result of a change in temperature from room temperature to body temperature. In this way, the tube could, for example, be inserted in one shape most convenient for insertion and would then change shape to one more suited to the anatomy when warmed to body temperature.

What I claim is:

1. A medico-surgical tube comprising: a shaft of a first material; and a separate member of a second, different material extending along a region of the length of said shaft, said second material being such that said tube can be bent by a user to a desired angle anywhere along said region with said tube retaining the angle of the bend.

2. A tube according to claim 1, wherein said second material is in the form of a rod of a malleable metal extending longitudinally along said region.

3. A tube according to claim 1, wherein said second material is a shape memory effect material.

4. A tube according to claim 1, wherein said region is spaced from a patient end of said tube.

5. A tube according to claim 1, wherein said region is spaced from a machine end of said tube.

6. A tube according to claim 1, wherein said first material is a plastics.

7. An endotracheal tube according to claim 1, wherein said region in use extends partly within and partly outside the body, and wherein said tube is bent at an angle where it emerges from the body such that a machine end of said tube extends substantially parallel with the patient's anatomy.

8. A tube according to claim 1, wherein said tube is extruded with a lumen within its wall, and wherein said second material extends along said lumen.

9. A tube according to claim 1, wherein said tube is curved along its length, and wherein said second material extends along an inside of the curve of said tube.

10. A tube according to claim 1, wherein said tube is reinforced with a helical reinforcement member.

11. An endotracheal tube comprising: a shaft of a plastics material; and a separate member of a malleable metal extending within a wall of said shaft along a region of the length of said shaft, said region being spaced from a patient end of said tube and extending where said tube in use extends through the mouth of the patient such that the tube can be bent by a user to a desired angle in the region of the mouth and retains the bend during use.
